(19) United States
(12) Patent Application Publication
(10) Pub. No.: US 2024/0428929 A1
(43) Pub. Date: Dec. 26, 2024

(54) AI-ASSISTED CLINICIAN CONTOUR REVIEWING AND REVISION

(71) Applicant: THE BOARD OF REGENTS OF THE UNIVERSITY OF TEXAS SYSTEM, Austin, TX (US)

(72) Inventors: Ti BAI, Austin, TX (US); Anjali BALAGOPAL, Austin, TX (US); Dan NGUYEN, Austin, TX (US); Steve JIANG, Austin, TX (US)

(73) Assignee: THE BOARD OF REGENTS OF THE UNIVERSITY OF TEXAS SYSTEM, Austin, TX (US)

(21) Appl. No.: 18/701,806

(22) PCT Filed: Oct. 18, 2022
(86) PCT No.: PCT/US2022/078251

§ 371 Date: Apr. 16, 2024

Related U.S. Application Data
(60) Provisional application No. 63/262,921, filed on Oct. 22, 2021.

(51) Int. Cl. G16H 30/40; G06T 7/00; G06T 7/12; G06T 7/50

(52) U.S. Cl. CPC G16H 30/40; G06T 7/0012; G06T 7/12; G06T 7/50; G06T 2207/20084; G06T 2207/20104

(57) ABSTRACT

Methods and systems for computer-assisted contour revision. An image slice may be selected from a medical image. The image slice may include an initial contour of a target anatomical structure in the medical image. At least a portion of the image slice and the initial contour may be displayed on a graphical user interface (GUI). Upon determining that the initial contour requires revision, a revised contour may be generated. A first input may be received from a user to the GUI to indicate a first point of revision. The medical image, the first input, and the initial contour may be input into a trained deep neural network that automatically extracts learned image characteristics. The extracted learned image characteristics may be processed using one or more deep-learning segmentation algorithms of the trained deep neural network. The revised contour may be automatically generated using the processed extracted learned image characteristics.

### CROSS REFERENCE TO RELATED APPLICATIONS

This application claims benefit to U.S. Provisional Application No. 63/262,921 entitled “AI-Assisted Clinician Contour Reviewing and Revision” filed Oct. 22, 2021. The full disclosure of this application is incorporated herein by reference.

### TECHNICAL FIELD

The present disclosure generally relates to radiation therapy, and more particularly to a novel system and method for using artificial intelligence (AI) based tools that can assist clinicians to quickly review and revise contours for targeted radiation treatment.

### BACKGROUND

Radiation therapy is one of the dominant ways to treat cancers and involves irradiating a tumor target volume of a patient with high-energy beams of photons, electrons, or heavy ions to a prescribed dose, while minimizing as much radiation dose to surrounding normal tissue and organs. Its success relies on the quality of the treatment plan, which is heavily dependent on multiple upstream processes prior to treatment planning. One of these processes is the process of target and organ segmentation. Accurate organ contouring is required, since low quality and inaccurate contours leads to damage to the surrounding tissue and organs and poor patient outcomes. However, conventional contouring processes are time-consuming and labor-intensive.

### SUMMARY

Methods and systems for computer-assisted contour revision are described herein. An image slice may be selected from a medical image of a patient. The image slice may include an initial contour of a target anatomical structure in the medical image. At least a portion of the image slice and the initial contour may be displayed on a graphical user interface (GUI). Upon determining that the initial contour requires revision, a revised contour may be generated. A first input may be received from a user to the GUI to indicate a first point of revision. The medical image, the first input, and the initial contour may be input into a trained deep neural network that automatically extracts learned image characteristics. The extracted learned image characteristics may be processed using one or more deep-learning segmentation algorithms of the trained deep neural network. The revised contour may be automatically generated using the processed extracted learned image characteristics.

### BRIEF DESCRIPTION OF THE DRAWINGS

The drawings described below are for illustration purposes only. The drawings are not intended to limit the scope of the present disclosure.

FIG. 1 is a flowchart illustrating an example workflow of an artificial intelligence-assisted contour revision (AIACR) process for contour revision, according to some embodiments of the present disclosure;

FIGS. 2A-2F are images illustrating initial contours and revised contours of a first dataset comprising images of a left parotid gland, according to some embodiments of the present disclosure;

FIGS. 3A-3F are images illustrating initial contours and revised contours of a second dataset comprising images of a left optical nerve, according to some embodiments of the present disclosure;

FIGS. 4A-4H are images illustrating initial contours and revised contours of a third dataset comprising images of a brainstem, according to some embodiments of the present disclosure;

FIGS. 5A-5B are charts illustrating a quantitative statistical analysis of the first dataset, according to some embodiments of the present disclosure;

FIGS. 6A-6B are charts illustrating a quantitative statistical analysis of the second dataset, according to some embodiments of the present disclosure;

FIGS. 7A-7B are charts illustrating a quantitative statistical analysis of the third dataset, according to some embodiments of the present disclosure;

FIG. 8 is a functional diagram illustrating modules used in the AIACR process, according to some embodiments of the present disclosure; and

FIG. 9 is a diagram of a system that may be used to perform the AIACR process, according to some embodiments of the present disclosure.

### DETAILED DESCRIPTION

Radiotherapy plays an important role in cancer patient treatment. Its success relies on elaborate treatment planning and radiation dose delivery to ensure sparing of the organs-at-risk (OAR) while delivering the prescription dose to the planning target volume (PTV). Upstream from the planning process is the segmentation step, which delineates the PTV and OARs. Since the segmentation result affects all downstream processes, accurate contours of the OARs and PTV are essential. For most of the cases in current clinical practice, the contouring is manually conducted by clinicians such that the final achieved contours can meet their demands. However, this process is quite labor-intensive and very time-consuming. Besides, manual contouring is sometimes impossible in some real-time-demanding scenarios, such as advanced online adaptive radiotherapy.

Existing computer aided tools, such as deep learning (DL) algorithms have been rapidly developed and deployed for automatic segmentation of organs and tumors in cancer radiotherapy. These methods typically concentrate on fully automating the contouring system by providing clinicians the automatically generated target and/or organ contours. The involvement of these tools in the clinical workflow stops after the automatic contours are generated and given to the clinicians. The segmentation results are not always perfect and thus require clinicians to manually review the contour for each organ in each image slice, revise it when needed, finally approve it, and assume liability for the clinical use of it.

Many resultant contours from conventional automated models still require manual revision to meet the clinically acceptable criterion because: 1) the trained models can never be perfect; 2) the DL-based automation models might be challenged by a well-recognized generalizability issue (i.e., the performance of a well-trained DL-based model might be degraded heavily when there exists distribution shift between the training and testing environments, such as different vendors and/or different institutes); and 3) different clinicians usually have their own contour style preferences even for the treatments of the same patient due to different clinical considerations based on their unique clinical experience. The latter scenario is extremely true for the clinical target volumes contouring where there are no clear boundaries. This preference should be respected for the clinical acceptance of the DL segmentation model. However, the manual revision process of DL-based automation models can be very time-consuming, sometimes requiring comparable time as the manual contouring from scratch, heavily hampering the clinical implementation of the automated DL-based segmentation models.

Therefore, a fast and accurate contouring process is desired to improve efficiency and accuracy in contour mapping for radiotherapy. The following disclosure includes systems and methods for artificial intelligence-assisted contour revision (AIACR) that addresses these challenges. As described in additional detail herein, an example workflow for AIACR may include, a user (e.g., a clinician) to indicate via user input to a device, where to make a revision to an automatically segmented organ or tumor contour. A well-trained DL model may take this input, along with the original medical image and any previously generated contours, to revise the contour. This process may repeat until an acceptable contour is achieved. The DL model is designed to minimize the clinician's inputs (at each iteration) and minimize the number of iterations needed to reach satisfaction.

The present disclosure will now be described more fully hereinafter with reference to the accompanying drawings, which form a part hereof, and which show, by way of non-limiting illustration, certain examples. Subject matter may, however, be described in a variety of different forms and, therefore, covered or claimed subject matter is intended to be construed as not being limited to any examples set forth herein. Among other things, subject matter may be described as methods, devices, components, or systems. Accordingly, examples may take the form of hardware, software, firmware or any combination thereof (other than software per se). The following detailed description is, therefore, not intended to be taken in a limiting sense.

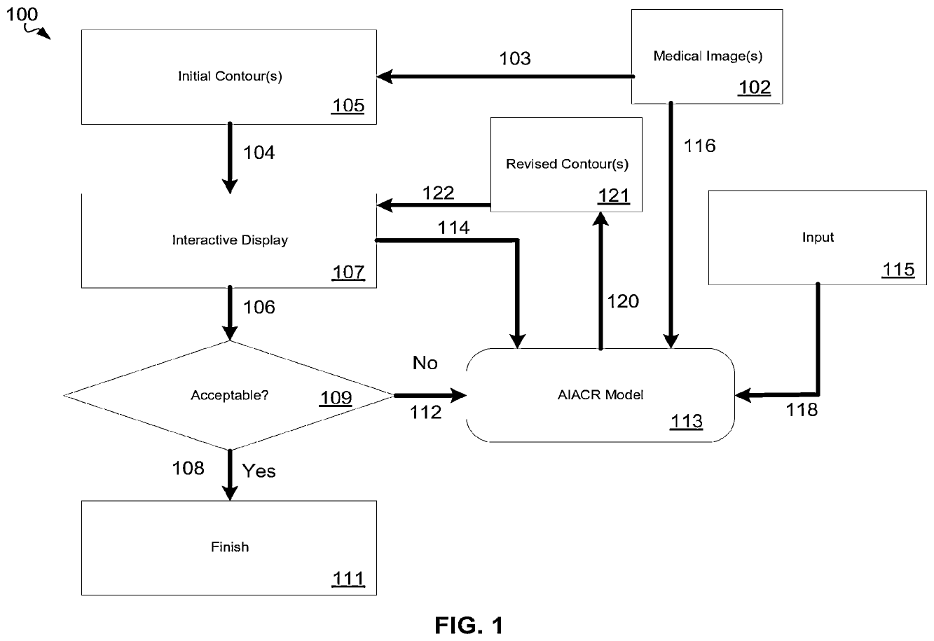

Referring now to FIG. 1, a flowchart illustrating an example workflow of an AIACR process 100 for contour revision is shown. The AIACR process 100 may begin with receiving one or more medical images 102. In step 103, the one or more medical images 102 may be processed to generate one or more initial contours 105. In an example, the one or more initial contours 105 may be generated for each of a respective one or more medical images 102. In another example, the one or more initial contours 105 may be generated for a portion of the one or more medical images 102.

The one or more initial contours 105 may be generated by any process, such as manual input via a user, a conventional automatic segmentation process, or a specially trained auto-segmentation module described below. For example, the one or more initial contours 105 may be generated by one or more of conventional graph cut algorithms, atlas-based segmentation algorithms, and registration-driven auto-segmentation algorithms.

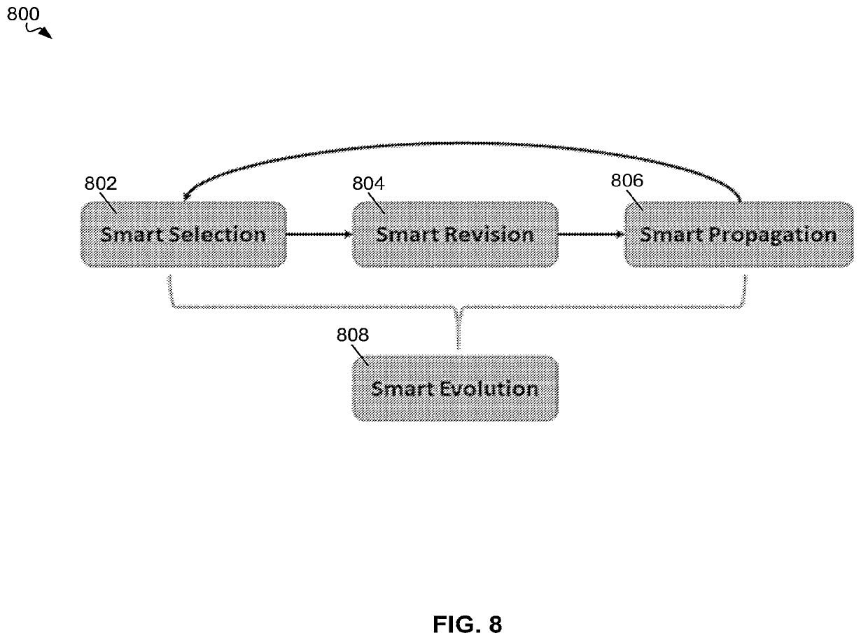

In step 104, at least a portion of the one or more of the initial contours 105 may be selected for review. In an example, the at least the portion of the one or more initial contours may be selected manually by a user. In another example, the at least the portion of the one or more initial contours may automatically be selected by a smart selection module 802 (described in detail below with reference to FIG. 8) that employs a DL model to determine the most valuable of the one or initial contours 105 for review.

The at least the portion of the initial contours 105 selected for review may be displayed to a user on an interactive display 107 of a user device (described herein). In step 106, the at least the portion of the initial contours 105 may be reviewed by a user to determine if they are acceptable 109. If the at least the portion of the initial contours 105 are acceptable, the process may continue to step 108 where the AIACR process 100 finishes 111. If any of the at least the portion of the initial contours 105, they may be selected for revision and the AIACR process 100 may continue to step 112, in which the AIACR model 113 is initiated.

In step 114, at least the selected one or more initial contours 105 may fed into the AIACR model 113. In an example, all of the at least the portion of the initial contours 105 may also be fed into the AIACR model 113. In another example, all of the one or more initial contours 105 may also be fed into the AIACR model 113. In step 116, the one or more medical images 102 may be fed into the AIACR model 113. In step 118, user input 115 may fed into the AIACR model 113. The user input 115 may be one or more interactions with the interactive display 107, such as a mouse click, a touch input to a touch-sensitive screen, a stylus touch input, etc. represent a single point of revision to the selected initial contour.

The AIACR model 113 may use one or more of the one or more medical images 102, the selected one or more initial contours 105, the at least the portion of the initial contours 105, the one or more initial contours 105, and the user input 115 as input into a machine learning algorithm. In step 120, the AIACR model 113 may generate one or more revised contours 121. In step 122, the one or more revised contours 121 may be sent to the interactive display 107 and the review/revision process may repeat until acceptable contours are achieved.

One or more of the auto-segmentation model, the smart selection module 802, and the AIACR model 113 may use one or more DL algorithms to learn information, process data, and revise results. In an example, one or more of the auto-segmentation model, the smart selection module 802, and the AIACR model 113 may use a U-Net architecture.

The U-Net architecture uses convolutional neural networks (CNN) for biomedical image segmentation. The U-Net architecture supplements a usual contracting network by successive layers, where pooling operations are replaced by upsampling operators. These layers increase the resolution of the output. What's more, a successive convolutional layer can then learn to assemble a precise output based on this information.

Another aspect of the U-Net architecture is that there are a large number of feature channels in the upsampling part, which allow the network to propagate context information to higher resolution layers. As a consequence, the expansive path is more or less symmetric to the contracting part, and yields a u-shaped architecture. The network only uses the valid part of each convolution without any fully connected layers. To predict the pixels in the border region of the image, the missing context is extrapolated by mirroring the input image. This tiling strategy is important to apply the network to large images, since otherwise the resolution would be limited by the GPU memory.

The network consists of a contracting path and an expansive path, which gives it the u-shaped architecture. The contracting path is a typical convolutional network that consists of repeated application of convolutions, each followed by a rectified linear unit (ReLU) and a max pooling operation. During the contraction, the spatial information is reduced while feature information is increased. The expansive pathway combines the feature and spatial information through a sequence of up-convolutions and concatenations with high-resolution features from the contracting path.

More specifically, in the encoder part, continuous stride-two convolutional layers may be applied to extract high-level features from the image. The feature number may be doubled per each downsampling operation from the stride-two convolutional operators until reaching a feature number of 512. The downsampling depth may be set as eight such that the bottleneck layer has a feature size of 1×1. In the decoder part, a concatenation operator may be adopted to fuse both low-level features and the high-level features. Regarding the network modules, each convolutional layer may contain three consecutive operators: convolution, instance normalization, and the ReLU. The convolutional operator may have a kernel size of 3×3.

The auto-segmentation model may have a single input channel and the AIACR model 113 may have three input channels. More specifically, the auto-segmentation model may receive the one or more medical images 102 in the first iteration as input. The AIACR model 113 may receive the selected one or more initial contours 105 (and in some examples all of the initial contours 105), the one or more medical images 102, and the user input 115 as inputs. The output channels for both models may be one. The initial feature numbers in the input layers of both models may be 64.

In general, the simpler the case is, the more accurate the initial generated contour is, and thus the higher chance the initial contour can be accepted. During the review process, the user may filter out difficult cases whose auto-generated initial contours are not acceptable and send them into the AIACR model 113 for further contour revision. As a guidance signal used by the AIACR model 113, the user may be asked to indicate where the selected contour should be revised.

As an assistant of the clinician, the goal of the AIACR model 113 is to minimize the user input 115 at each iteration and minimize the number of iterations required. In an example, the user input 115 for each iteration may be a single interaction (e.g., one mouse click, one touch input to a touch-sensitive screen, one stylus touch input) on a boundary point for efficient and controllable contour revision. The user may preferentially revise a contour segment with large errors since it allows less iterations. The AIACR model 113 may greatly improve the healthcare providing efficiency and promote the clinical application of the AI models in practice.

In an example, the user input 115 at each iteration may be a single interaction (e.g., one mouse click) on a desired location of a selected contour segment which has the largest error at the current iteration. The performance of the AIACR model 113 may be measured by a percentage of contours that meet the criterion 95th percentile of Hausdorff Distance (HD95)<2.5 mm as well as the number of discrete user input 115 needed to reach the criterion.

In an example, the one or more medical images 102 may be two-dimensional (2D). To be compatible with the dimension of the one or more medical images 102, the user input 115 may be converted into a 2D image (i.e., click image) by placing a 2D Gaussian point with a radius (e.g., 10 pixels) around the selected boundary point.

The clinician may further review the one or more revised contours 121. If the one or more revised contours 121 are acceptable, this revision process may be terminated. Otherwise, a second boundary point that exhibits large errors will be clicked. In this case, both the first and the second mouse clicks are simultaneously converted into a single 2D click image by placing two 2D Gaussian points (both has a radius of 10 pixels) around the above two boundary points. The updated click image will be feed into the AIACR model 113 for a further revision of the contour. This process can be repeated by a few iterations (clicks) until the resultant contour matches the region intended by the clinician.

One or more of the auto-segmentation model and the AIACR model 113 may be trained using a first dataset. In an example, open access head & neck (HN) CT scans of nasopharynx cancer patients from the “Automatic Structure Segmentation for Radiotherapy Planning Challenge” were used as a training dataset. Each CT scan in the first dataset was marked by one experienced oncologist and verified by another experienced one. The first dataset was randomly split into 40 and 10 patients to serve as training and validation datasets, respectively. Twenty-one (21) annotated OARs were employed for the performance quantification.

The original CT scans in the first dataset consisted of approximately 100-200 slices of 512×512 pixels, with a voxel resolution of ([0.98˜1.18]×[0.98˜1.18]×3.0 mm³). Since 2D slices of CT images were used, this resulted in 4941 images for training and 1210 images for validation.

In an example, the AIACR model 113 was trained based on the above first dataset using one or more of Dice loss and Hausdorff distance (HD)-based loss. A weight was used to balance these two losses such that the weighted HD-based loss had values similar to the Dice loss for any training sample. Adam optimization was used with parameters β₁=0.9 and β₂=0.999 to update the model parameters at every 1×10⁵ iterations. In an example, the learning rate was initially set as 1×10⁻⁴, which was then reduced to 1×10⁻⁵ and 1×10⁻⁶ at iterations 5×10⁴ and 7.5×10⁴, respectively. The batch size was one.

The auto-segmentation model may be trained using similar methods. In an example, the same Dice loss and HD-based loss were used for the model training. A weight was used to balance these two losses such that the weighted HD-based loss had the same value as the Dice loss for any training sample. Other training details for the auto-segmentation model (e.g., optimizer, learning rate policy and batch size) were the same as the auto-segmentation model training.

It should be noted that interactive segmentation datasets with real clinician's feedback signals are not available. Indeed, it is very hard, if not impossible, to collect the clinician's revision action for an existing contour. Therefore, an interactive segmentation dataset was constructed during the training phase based on the above training dataset by reasonably simulating the clinician's potential action for the contour revision given current segmentation map (e.g., the highest chance to be the desired boundary point with the largest distance from the current contour).

Given a current predicted contour (denoted as Cₚ), the distance between each point on the ground truth contour C_{g} to current predicted contour Cₚ was calculated as below:

$${D}_{{C}_{p}} ( y ) = {inf}_{x ∈ {C}_{p}} d ( x , y ) ∀ y ∈ {C}_{g} , \qquad Equation 1$$

where d(x,y) represents the distance between the points on Cₚ (denoted as x) and the points on C_{g} (denoted as y). In other words, the above function, D_{C}_{ₚ}(y), represents the distance for every point on the ground truth contour to the nearest point on the predicted contour.

Assuming that the clinician would have higher probability to click on those points on the ground truth contour that have large errors, the distance measurement may be converted to click probability. Based on the above calculated distances, a SoftMax transformation was used to assign a click probability to each ground truth boundary point as follows:

$$P ( y ) = \frac{exp ( - {D}_{{C}_{p}} ( y ) )}{{\sum }_{{y}^{′} ∈ {C}_{g}} exp ( - {D}_{{C}_{p}} ( {y}^{′} ) )} ∀ y ∈ {C}_{g} \qquad Equation 2$$

Given the current segmentation map, the clinician's click point on the ground truth contour was randomly sampled based on the probabilistic distribution defined by Equation 2. The larger the error of some specific point on the ground truth contour, the larger the distance associated with that point defined by Equation 1 is, and consequently, the higher chance that point will be sampled (clicked by the clinician).

Given the above randomly sampled point, the current segmentation map, and the input CT image, three-channel input data was constructed and fed into the AIACR model 113 for training. The supervised signal may be the same as that of the auto-segmentation model training (i.e., the ground truth segmentation mask).

To demonstrate the AIACR model's 113 generalizability to data from previously unseen demographics and distributions, a second dataset and a third dataset were employed for further performance verification.

The second dataset consisted of deidentified HN CT scans which were initially segmented with full volumetric regions by a radiographer with at least four years' experience and then arbitrated by a second radiographer with similar experience. Further arbitration was then performed by a radiation oncologist with at least five years' post-certification experience.

In the second dataset, there were 7 scans for validation and 28 scans for testing. Each scan in the second dataset had 21 annotated OARs and consisted of 119˜184 slices of 512×512 pixels, with a voxel resolution of ([0.94˜1.25]×[0.94˜1.25]×2.50 mm³. Twenty-eight (28) scans consisting of 4427 2D slice CT images were used for performance testing. Ten (10) of the 21 OARs were overlapped with the first dataset, and thus used to quantify the performance.

The third dataset contained 20 patient scans. The contours of the OARs were exported from a clinical system and cleaned with in-house-developed programs followed by a manual double-check. Each scan had at most 53 annotated OARs. Depending on different clinical tasks, different patients may have different OARs annotated. In the third dataset, the scans contained 124˜203 slices of 512×512 pixels, with a voxel resolution of 1.17˜1.37]×[1.17˜1.37]×3.00 mm³. In total, 2980 2D slice CT images were included in the third dataset. Ten (10) OARs overlapped with the first dataset were used for algorithm's evaluation.

For computational efficiency consideration, for each volumetric CT scan in the training, validation and testing datasets, a sub-volume with an axial size of 256×256 was cropped out such that the whole HN regions are covered.

To deterministically quantify the performance of the AIACR model 113 during the testing phase, without loss of generality, it was assumed that the clinician will always click on the point with the largest error (i.e., the largest distance defined by Equation 1, which is the one-side HD from the ground truth contour C_{g} to the current predicted contour Cₚ). Two different metrics were used for the model performance quantification: Dice coefficient and the HD95.

Assuming the maximum number of the allowed clicks is 20, the criterion for a contour that can be accepted in clinics was set as HD95<2.5 mm, which can meet the precision demands for most of the radiotherapy treatment tasks. The percentage of the acceptable contours after revision was calculated with the aid of our AIACR model 113, along with the median number of clicks required to reach this acceptability. As an overall quantification, the averaged values of the above two metrics were computed among all the investigated OARs separately in the validation, DeepMind, and UTSW testing datasets. The model inference efficiency (i.e., the response time for contour updating after the clinician performs a click) was also reported.

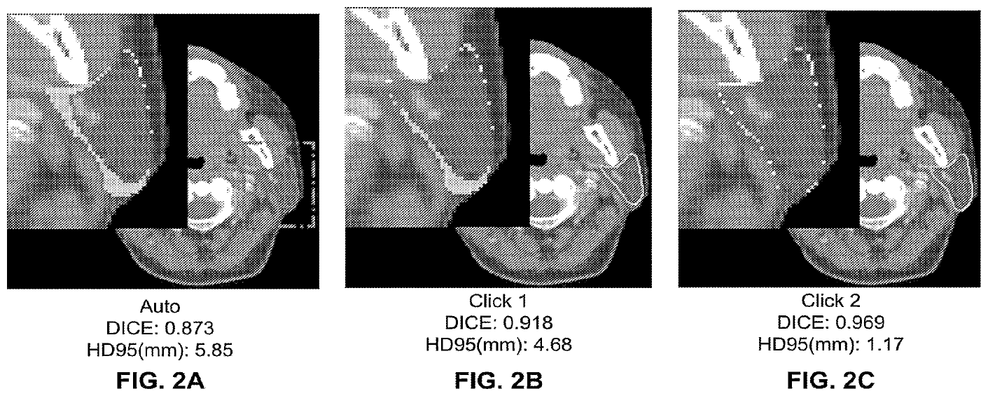

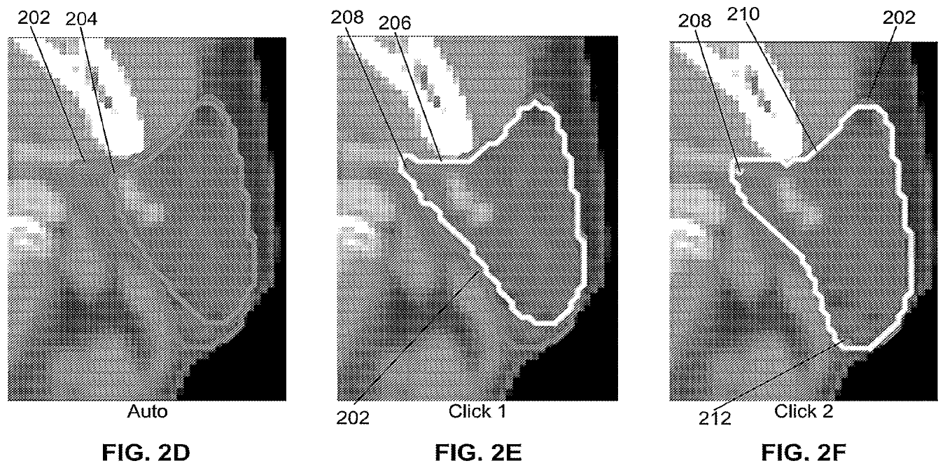

Referring now to FIGS. 2A-2F, images illustrating an initial contour 105 and revised contours 121 generated by the AIACR process 100 using medical images 102 of a left parotid gland (i.e., the first dataset) are shown.

FIG. 2A shows an initial contour 105 generated by the auto-segmentation model. FIG. 2B shows a revised contour 121 generated by the AIACR model 113 after a single iteration (i.e., one instance of user input 115). FIG. 2C shows a revised contour 121 generated by the AIACR model 113 after a second iteration (i.e., two total instances of user input 115).

FIG. 2D shows a zoomed-in comparison between a ground truth contour 202 and an auto-contour 204 generated by auto-segmentation model. FIG. 2E shows a zoomed in comparison of a first revised contour 206, the ground truth contour 202, and a first point 208 representing a first user input 115. As shown in FIG. 2E, the first revised contour 206 tracks closer to the first point 208 than the auto-contour 204. FIG. 2F shows a zoomed in comparison of a second revised contour 210, the ground truth contour 202, the first point 208 representing the first user input 115, and a second point 212 representing a second user input 115. The second point 212 may be represented as a different color than the first point 208. As shown in FIG. 2F, the second revised contour 210 tracks closer to the second point 212 than the first revised contour 206. The segmentation errors are also demonstrated in the top left corner of FIGS. 2A-2C (e.g., green is a false negative and yellow is false positive).

As shown in FIG. 2D, there is a large gap between the ground truth contour 202 and the initial auto-contour 204. After the first user input 115 (e.g., a single mouse click) shown as the first point 208, most parts of the first revised contour 206 around that clicked point are corrected. The associated Dice/HD95 losses are improved from 0.873/5.85 in the auto-contour 204 to 0.918/4.68 in the first revised contour 206. After a second iteration and the second user input 115 (e.g., another single mouse click) shown as the second point 212, the second revised contour 210 matches well with the ground truth contour 2020, with Dice/HD95 losses of 0.969/1.17. This gradual improvement with each user input 115 may be also observed in the segmentation error maps shown in FIGS. 2A-2C, where substantial false negative errors are corrected. The segmentation error map associated with two iterations of user input 115 shown in FIG. 2C, shows an only one-pixel error between the second revised contour 210 and the ground truth contour 202.

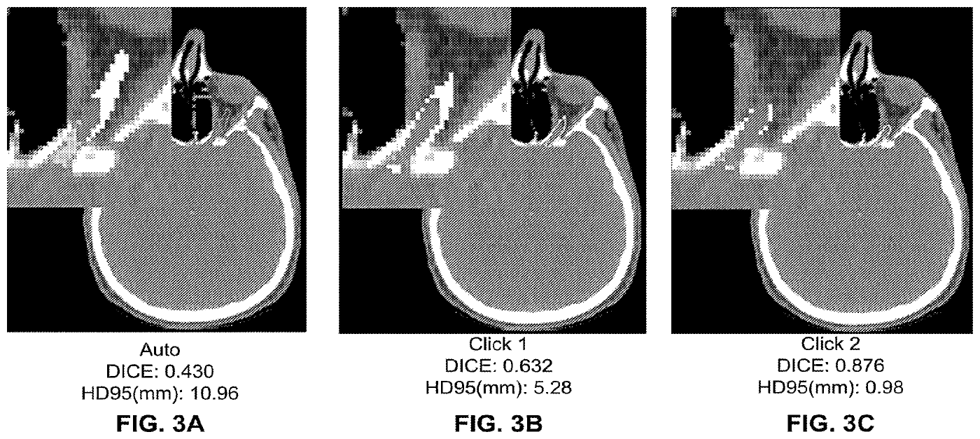

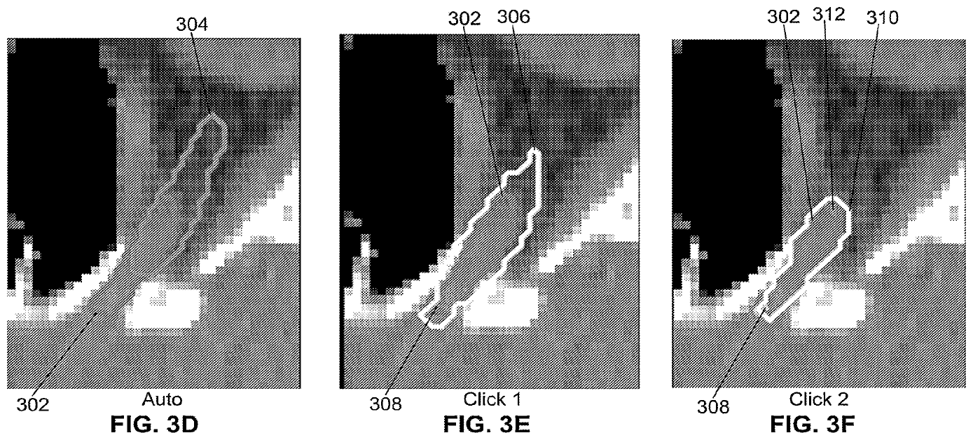

Referring now to FIGS. 3A-3F, images illustrating an initial contour 105 and revised contours 121 generated by the AIACR process 100 using the medical images 102 of a left optical nerve (i.e., the second dataset) are shown.

FIG. 3A shows an initial contour 105 generated by the auto-segmentation model. FIG. 3B shows a revised contour 121 generated by the AIACR model 113 after a single iteration (i.e., one instance of user input 115). FIG. 3C shows a revised contour 121 generated by the AIACR model 113 after a second iteration (i.e., two total instances of user input 115).

FIG. 3D shows a zoomed-in comparison between a ground truth contour 302 and an auto-contour 304 generated by the auto-segmentation model. FIG. 3E shows a zoomed in comparison of a first revised contour 306, the ground truth contour 302, and a first point 308 representing a first user input 115. As shown in FIG. 3E, the first revised contour 306 tracks closer to the first point 308 than the auto-contour 304. FIG. 3F shows a zoomed in comparison of a second revised contour 310, the ground truth contour 302, the first point 308 representing the first user input 115, and a second point 312 representing a second user input 115. The second point 312 may be represented as a different color than the first point 308. As shown in FIG. 3F, the second revised contour 310 tracks closer to the second point 312 than the first revised contour 306. The segmentation errors are also demonstrated in the top left corner of FIGS. 3A-3C (e.g., green is a false negative and yellow is false positive).

As shown in FIGS. 3D-3F, a good match between the second revised contour 310 and the ground truth contour 302 can be achieved with two instances of user input 115 (e.g., two mouse clicks). The error map of FIG. 3C shows that the differences between the second revised contour 310 and the ground truth contour 302 may be within one pixel. By contrast, the error map of the auto-contour 304 shown in FIG. 3A demonstrates large false positive and negative errors despite this optical nerve exhibits a clear boundary. Quantitatively, after two instances of user input 115, the DICE/HD95 loss metrics are dramatically improved from 0.43/10.96 for the auto-contour 304 to 0.876/0.98 for the second revised contour 310.

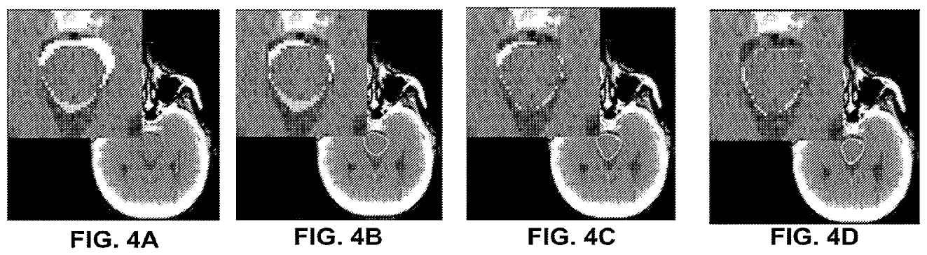

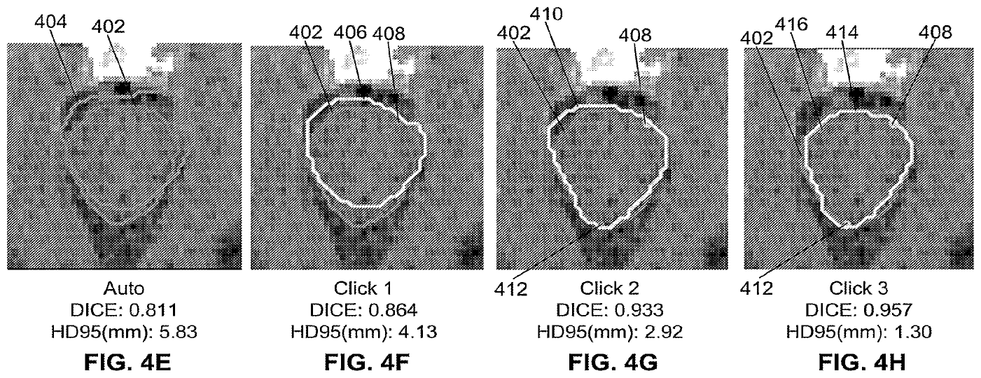

Referring now to FIGS. 4A-4H, images illustrating an initial contour 105 and revised contours 121 generated by the AIACR process 100 using medical images 102 of a brainstem (i.e., the third dataset) are shown.

In this example, three iterations (and three instances of user input 115) were required to reach the preset HD95<2.5 mm threshold. As shown in FIG. 4A, the initial contour 105 from the auto-segmentation model shows great over/under segmentation errors than the above examples, especially in the anterior direction.

FIG. 4A shows an initial contour 105 generated by the auto-segmentation model. FIG. 4B shows a revised contour 121 generated by the AIACR model 113 after a single iteration (i.e., one instance of user input 115). FIG. 4C shows a revised contour 121 generated by the AIACR model 113 after a second iteration (i.e., two total instances of user input 115). FIG. 4D shows a revised contour 121 generated by the AIACR model 113 after a third iteration (i.e., three total instances of user input 115).

FIG. 4E shows a zoomed-in comparison between a ground truth contour 402 and an auto-contour 404 generated by auto-segmentation model. FIG. 4F shows a zoomed in comparison of a first revised contour 406, the ground truth contour 402, and a first point 408 representing a first user input 115. As shown in FIG. 4F, the first revised contour 406 tracks closer to the first point 408 than the auto-contour 404. FIG. 4G shows a zoomed in comparison of a second revised contour 410, the ground truth contour 402, the first point 408 representing the first user input 115, and a second point 412 representing a second user input 115. The second point 412 may be represented as a different color than the first point 408. As shown in FIG. 4G, the second revised contour 410 tracks closer to the second point 412 than the first revised contour 406. FIG. 4H shows a zoomed in comparison of a third revised contour 414, the ground truth contour 402, the first point 408 representing the first user input 115, the second point 412 representing a second user input 115, and a third point 416 representing a third user input 115. The third point 416 may be represented as a different color than the first point 408 and the second point 412. As shown in FIG. 4H, the third revised contour 414 tracks closer to the third point 416 than the second revised contour 410. The segmentation errors are also demonstrated in the top left corner of FIGS. 4A-4D (e.g., green is a false negative and yellow is false positive).

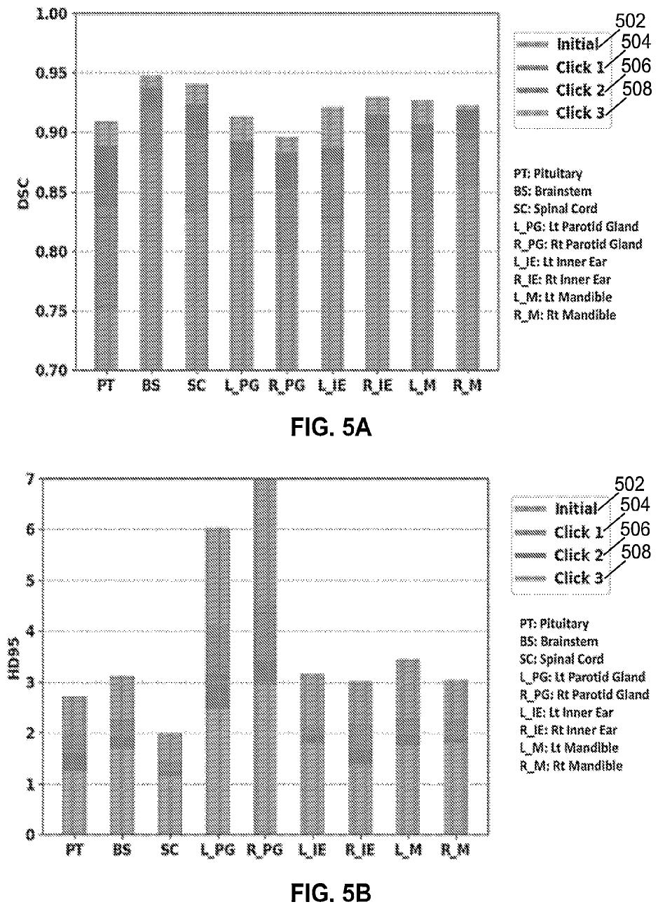

Referring now to FIGS. 5A-5B, charts illustrating a quantitative statistical analysis of the first dataset are shown. FIGS. 5A-5B show a gradual performance improvement from an initial contour 502 to each round of user input 115 (e.g., a first click 504, a second click 506, and a third click 508) based on selected clinically significant and relatively challenging organ cases. The y-axis in FIG. 5A shows the Dice coefficients and the y-axis in FIG. 5B shows the HD95 coefficients, which may be used to quantify the performance improvement for three datasets. The x-axis represents different organs, whose abbreviations are listed to the right of the charts.

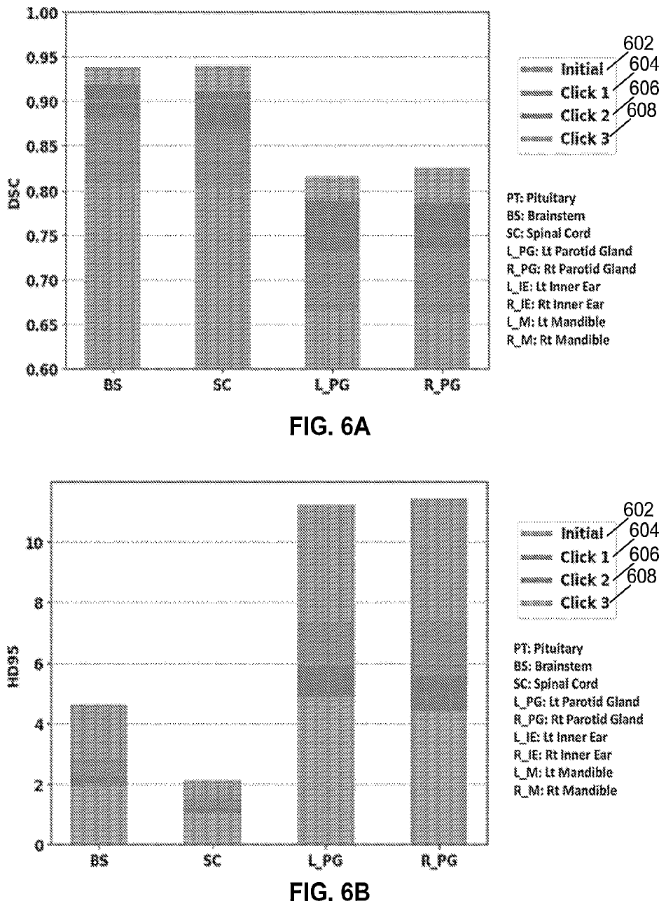

Referring now to FIGS. 6A-6B, charts illustrating a quantitative statistical analysis of the second dataset are shown. FIGS. 6A-6B show a gradual performance improvement from an initial contour 602 to each round of user input 115 (e.g., a first click 604, a second click 606, and a third click 608) based on selected clinically significant and relatively challenging organ cases. The y-axis in FIG. 6A shows the Dice coefficients and the y-axis in FIG. 6B shows the HD95 coefficients, which may be used to quantify the performance improvement for three datasets. The x-axis represents different organs, whose abbreviations are listed to the right of the charts.

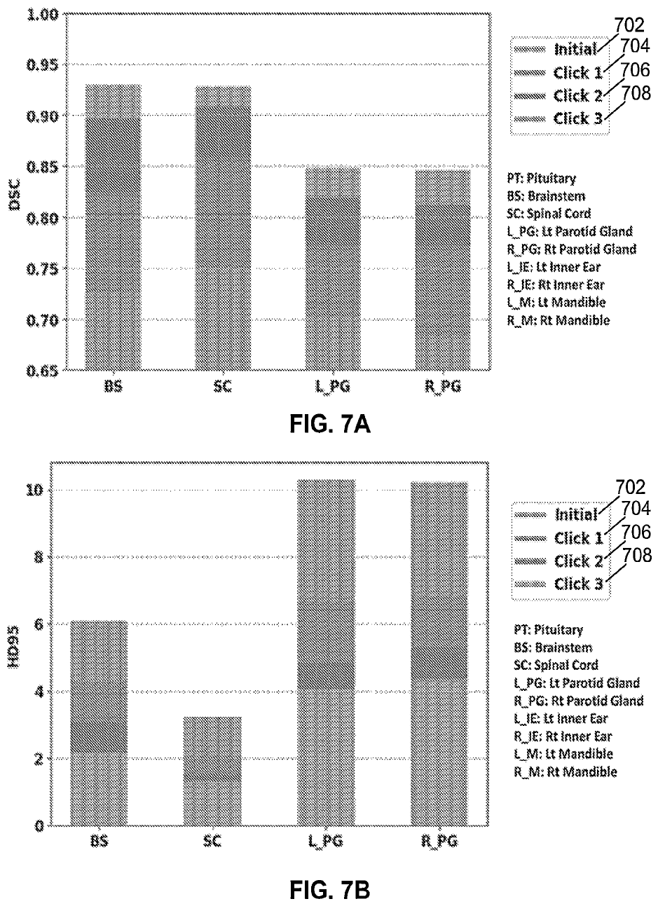

Referring now to FIGS. 7A-7B, charts illustrating a quantitative statistical analysis of the third dataset are shown. FIGS. 7A-7B show a gradual performance improvement from an initial contour 702 to each round of user input 115 (e.g., a first click 704, a second click 706, and a third click 708) based on selected clinically significant and relatively challenging organ cases. The y-axis in FIG. 7A shows the Dice coefficients and the y-axis in FIG. 7B shows the HD95 coefficients, which may be used to quantify the performance improvement for three datasets. The x-axis represents different organs, whose abbreviations are listed to the right of the charts.

TABLE 1
DSC/HD95 for Datasets

| | First Dataset | Second Dataset | Third Dataset |
|---|---|---|---|
| Initial | 0.82/4.3 | 0.73/5.6 | 0.67/11.4 |
| Click 1 | 0.87/3.0 | 0.78/3.6 | 0.76/7. 5 |
| Click 2 | 0.89/2.4 | 0.83/2.8 | 0.82/5.7 |
| Click 3 | 0.91/2.1 | 0.86/2.4 | 0.86/4.7 |

Table 1 illustrates the performance improvement with three clicks quantified by the averaged Dice Coefficients (DSC) and HD95 among all the organs for the different datasets.

Overall, Table 1 shows a more than 10 percent absolute increase in DSC on all three datasets after three clicks, while HD95 was cut almost in half. Moreover, the poorer the initial performance (UTSW dataset), the greater the improvement that AIACR process 100 achieved. In addition, the time required to update the contour per each user input 115 is approximately 20 ms if using a single NVIDIA GeForce Titan X graphics card, which allows a user to interact in real time during the contour revision process.

The AIACR process 100 may assist clinicians in making decisions and may make performing clinical procedures better and faster, not by replacing the clinician with a fully automated process, by working with the clinician and making intelligent decisions based on machine learning.

The AIACR process 100 may include two different models for contour generation: the auto-segmentation model for initial contour generation and the AIACR model 113 for faster contour revision through interactions with the clinician. The clinician may first review the initial contour generated by the auto-segmentation model, which may be accepted as is (AAI), accepted with revision (AWR), or rejected (REJ). If a revision is required, the AIACR model 113 may be used to aid the clinician. The goal of the auto-segmentation model may be to maximize the AAI ratio and minimize the REJ ratio, while the goal of the AIACR model 113 may be to improve the revision efficiency for those cases that are AWR.

The efficiency improvement (e.g., the amount of user input 115 required to revise the contour given a preset criterion) of the AIACR model 113 may depend on the performance of the auto-segmentation model. Given an auto-generated initial contour, it is expected that the closer it is to the desired contour, the less user input 115 may be required during the revision process.

Referring now to FIG. 8, a functional diagram 800 illustrating modules that may be used in the AIACR process 100 is shown. In an example, the AIACR process 100 may use one or more of: a smart selection module 802, a smart revision module 804, a smart propagation module 806, and a smart evolution module 808.

The smart selection module 802 may minimize the effort devoted by the clinician during the process of reviewing image slices and contours and thereby selecting the most valuable slices that should be revised in the next stage.

As described above, the smart selection module 802 may be used in step 104, in which 104, at least a portion of the one or more of the initial contours 105 may be selected for review. The smart selection module 802 may include a DL model to analyze the information extracted from the input one or more medical images 102, the one or more of the initial contours 105, the revision histories of each, and/or the uncertainty of the contours, and/or the contour quality. The DL model may be the same or similar to the DL model used in the auto-segmentation model and the AIACR model 113 described above. The DL model may intelligently select and output one or more slice indexes that should be revised in the next stage. In an example, the smart selection module 802 may output only a portion of the one or more of the initial contours 105 for review.

Both the uncertainty and the quality of the contours may be predicted with the same DL model or another DL model. The smart selection module 802 may be enabled or disabled. In the enabled mode, the smart selection module 802 may smartly select the slice for revision recommendation. In the disabled mode, the clinician may choose the slices to be revised based on their own expertise.

The smart revision module 804 may be employed by the AIACR model 113 and may minimize the effort made by the clinician to revise a contour. As described above, the smart revision module 804 may also be built upon DL techniques and may function in 2D and/or 3D modes. Given a contour (either selected automatically or manually by a user), the clinician may enter user input 115 (e.g., a click, a touch, and/or drawing a scribble) to indicate where the contour should be revised. Then, the smart revision module 804 may gather information including the one or more medical images 102 and the feedbacked revision signal for analyzing and update the contour which should be closer to the ground truth contour. During this revision process, the uncertainty map of the current contours may also be provided to the clinician for suggestion about the potential region that should be revised.

The smart propagation module 806 may maximize the interpolation accuracy of the contours of those untouched slices, given the reliable revised contours. The smart propagation module 806 may be built upon DL techniques. The DL model used by the smart propagation module 806 may be the same or similar to the DL model used in the auto-segmentation model and the AIACR model 113 described above The smart propagation module 806 may take as inputs one or more of the one or more medical images 102, the current contours, the one or more revised contours 121, the contour quality, and the segmentation uncertainty. After analyzing these information, the smart propagation module 806 may update the contours, and/or the contour qualities, and/or the segmentation uncertainties. The smart propagation module 806 may take any revisions made to one or more initial contours 105, which may be only on a portion of the one or more medical images 102, and may extrapolate those revisions to generate (i.e., propagate) contours and/or revise contours on the remaining one or more medical images 102.

The smart evolution module 808 may maximize overall system performance by collecting more data for updating the one or more DL models described above in an online/offline fashion. The smart evolution module 808 may automatically collect and clean data, such as previously analyzed medical images 102 and previously generated initial contours 105 and revised contours 121. Based on the newly added data, the smart evolution module 808 may update one or more of the auto-segmentation model, the AIACR model 113, the smart selection module 802, the smart revision module 804, and the smart propagation module 806 to increase performance. The smart evolution module 808 may provide the update a customized time interval, such as daily/weekly/monthly. When the one or more models are update, hyper-parameters may automatically be selected using machine learning techniques.

It should be noted that each of these modules may be used interpedently of the AIACR process 100. The modules may work independently with other existing tools or they may work together for maximum efficiency boosting.

The AIACR process 100 directly addresses a problem that most AI models face when deployed into clinical practice. Conventional techniques for implementing AI clinically typically involves AI and clinicians working independently and sequentially. For example, the AI may independently perform a clinical task (e.g., segmentation) and presents the outcome to clinicians to review. The clinicians may then accept, reject, or revise the outcome. In contrast, the AIACR process 100 may integrate AI into clinicians' existing workflows, allowing them to perform clinical tasks collaboratively to achieve a clinically acceptable result in a more efficient and user-friendly manner. This may facilitate clinicians' acceptance of AI being implemented into routine clinical practice.

For the clinical task of organ and tumor segmentation (e.g., after the auto-segmentation model generates an initial contour) the clinician may face three options: accept as is (AAI), accept with revision (AWR), and reject (REJ). The accuracy of the auto-segmentation model may be increased to maximize the AAI ratio and minimize the REJ ratio. If the auto-segmentation model produced a 100% AAI ratio, the AI may be able to independently finish the clinical task without clinicians' inputs, which would potentially allow AI to replace clinicians. However, in practice, auto-segmentation models may not be able to achieve a 100% AAI ratio, especially for challenging cases, and clinicians' manual revisions of the initial contours may be always needed. Accordingly, the AIACR process 100 described herein may assist clinicians in revising the initial contours in an efficient and user-friendly way. The AIACR process 100 may not replace auto-segmentation models. Instead, it may work downstream of an auto-segmentation model, so it may be used with any state-of-the-art auto-segmentation model.

One of the advantages of the AIACR process 100 is that it may alleviate the generalizability issue from which many DL-based auto-segmentation models suffer. Even if an auto-segmentation model generalizes poorly to outside datasets, using the AIACR process 100 may greatly improve these results, as shown in Table 1.

The AIACR process 100 may play an important role in online adaptive radiotherapy (ART). The current pipeline for ART includes acquiring a same day image, such as a cone beam CT (“CBCT”), and deforming or creating new OARs and target structures to optimize the radiation treatment plan, given the patient's current anatomy. This approach may account for gas passing through the intestines or tumors shrinking from current therapy. However, a significant barrier to implementing online ART is the time required to manually correct contours. Therefore, improving the efficiency of clinicians' contour revision via the AIACR process 100 may improve the acceptance of online ART by radiotherapy departments.

Several online ART modalities are currently being investigated clinically, including CBCT-based, MRI-based, and PET/CT-based techniques. Using conventional CBCT-based adaptive approaches for head-and-neck cancer as an example, the median time spent in this virtual online ART process may be almost 20 minutes, with a range of 13 to 31 minutes. Most of the most time may be spent reviewing and editing contours. The AIACR process 100 may reduce the time spent reviewing and editing contours and this adaptive process may be more easily integrated into the already strained time requirements of a busy practice.

In should be noted that 2D axial images were used herein to demonstrate the feasibility of the AIACR process 100. However, the techniques described herein may also apply to 3D cases.

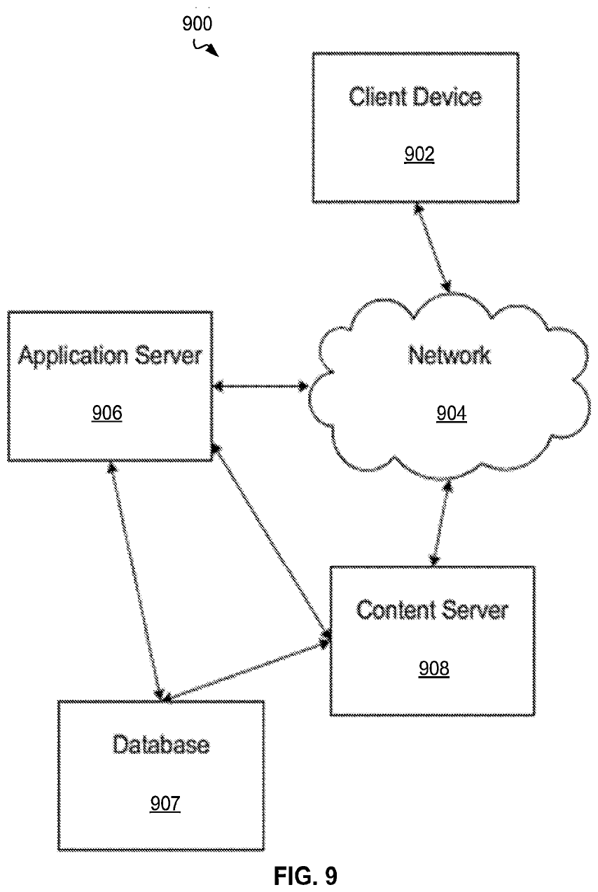

Referring now to FIG. 9, a system 900 is shown. FIG. 9 illustrates components of a general environment in which the systems and methods discussed herein may be practiced. Not all the components may be required to practice the disclosure, and variations in the arrangement and type of the components may be made without departing from the spirit or scope of the disclosure.

The system 900 of FIG. 9 includes network 904, which as discussed above, may include, but is not limited to, a wireless network, a local area network (LAN), wide area network (WAN), the Internet, or a combination thereof.

The network 904 may be connected, for example, to one or more client devices 902, an application server 906, a content server 908, and a database 907 and their components with another network or device. The network 904 may be configured as a variety of wireless sub-networks that may further overlay stand-alone ad-hoc networks, and the like, to provide an infrastructure-oriented connection for the one or more client devices 902, the application server 906, the content server 908, and the database 907. The network 904 may be configured to employ any form of computer readable media or network for communicating information from one electronic device to another.

The one or more client devices 902 may, for example, include a desktop computer or a portable device, such as a cellular telephone, a smart phone, a display pager, a radio frequency (RF) device, an infrared (IR) device, a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

The one or more client devices 902 may also include at least one client application that is configured to receive content from another computing device. The one or more client devices 902 may communicate over the network 904 with other devices or servers, and such communications may include sending and/or receiving messages, generating and providing TCR data, searching for, viewing and/or sharing TCR data, or any of a variety of other forms of communications. The one or more client devices 902 may be capable of processing or storing signals, such as in memory as physical memory states, and may, therefore, operate as a server

The application server 906 and the content server 908 may include one or more devices that are configured to provide and/or generate any type or form of content via a network to another device. Devices that may operate as the application server 906 and/or the content server 908 may include personal computers, desktop computers, multiprocessor systems, microprocessor-based or programmable consumer electronics, network PCs, servers, and the like. The application server 906 and the content server 908 may store various types of data related to the content and services provided by each device in the database 907.

Users (e.g., patients, doctors, technicians, and the like) may be able to access services provided by the application server 906 and the content server 908. This may include, for example, application servers, authentication servers, search servers, exchange servers, via the network 904 using the one or more client devices 902. Thus, the application server 906, for example, may store various types of applications and application related information including application data and user profile information.

Although FIG. 7 illustrates the application server 906 and the content server 908 as single computing devices, respectively, the disclosure is not so limited. For example, one or more functions of the application server 906 and the content server 908 may be distributed across one or more distinct computing devices. In another example, the application server 906 and the content server 908 may be integrated into a single computing device without departing from the scope of the present disclosure.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described with reference to block diagrams and operational illustrations of methods and devices. It is understood that each block of the block diagrams or operational illustrations, and combinations of blocks in the block diagrams or operational illustrations, may be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession may be executed substantially concurrently or the blocks may sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, cloud storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5^{th} generation (2G, 3G, 4G or 5G) cellular technology, Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For the purposes of this disclosure a module is a software, hardware, or firmware (or combinations thereof) system, process or functionality, or component thereof, that performs or facilitates the processes, features, and/or functions described herein (with or without human interaction or augmentation). A module may include sub-modules. Software components of a module may be stored on a computer readable medium for execution by a processor. Modules may be integral to one or more servers, or be loaded and executed by one or more servers. One or more modules may be grouped into an engine or an application.

Those skilled in the art will recognize that the methods and systems of the present disclosure may be implemented in many manners and as such are not to be limited by the foregoing examples. In other words, functional elements being performed by single or multiple components, in various combinations of hardware and software or firmware, and individual functions, may be distributed among software applications at either the client level or server level or both. In this regard, any number of the features of the different examples described herein may be combined into single or multiple examples, and alternate examples having fewer than, or more than, all of the features described herein are possible.

Functionality may also be, in whole or in part, distributed among multiple components, in manners now known or to become known. Thus, a myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the examples of methods presented and described as flowcharts in this disclosure are provided by way of example in order to provide a more complete understanding of the technology. The disclosed methods are not limited to the operations and logical flow presented herein. Alternative examples are contemplated in which the order of the various operations is altered and in which sub-operations described as being part of a larger operation are performed independently.

While various examples have been described for purposes of this disclosure, such examples should not be deemed to limit the teaching of this disclosure to those examples. Various changes and modifications may be made to the elements and operations described above to obtain a result that remains within the scope of the systems and processes described in this disclosure.

What is claimed is:

1. A method for computer-assisted contour revision in medical image segmentation, comprising:
selecting an image slice from one or more medical images of a patient, the image slice comprising an initial contour of a target anatomical structure in the one or more medical images;
displaying at least a portion of the image slice and the initial contour on a graphical user interface (GUI); and
upon determining that the initial contour requires revision, generating a revised contour by:
receiving a first input from a user to the GUI to indicate a first point of revision,
inputting the one or more medical images, the first input, and the initial contour into a trained deep neural network that automatically extracts learned image characteristics,
processing the extracted learned image characteristics using one or more deep-learning segmentation algorithms of the trained deep neural network, and
automatically generating the revised contour using the processed extracted learned image characteristics.

2. The method of claim 1, wherein the one or more medical images comprise three-dimensional (3D) images.

3. The method of claim 1, wherein the image slice comprises a two-dimensional (2D) image.

4. The method of claim 1, wherein the initial contour is generated by an external system.

5. The method of claim 1, further comprising:
inputting the one or more medical images into the trained deep neural network; and
automatically generating the initial contour using the one or more deep-learning segmentation algorithms to process the extracted learned image characteristics.

6. The method of claim 5, further comprising:
automatically generating one or more of an uncertainty value and a quality value for the initial contour.

7. The method of claim 6, wherein the selecting the image slice is done automatically based on, at least, one or more of the one or more of the uncertainty value and the quality value.

8. The method of claim 6, further comprising:
displaying the one or more of the uncertainty value and the quality value on the GUI.

9. The method of claim 1, further comprising:
displaying the one or more medical images on the GUI; and
receiving user input to the GUI to generate the initial contour.

10. The method of claim 1, wherein the selecting the image slice is done manually via user input to the GUI.

11. The method of claim 1, wherein the first input comprises one or more of a single mouse click and a touch input to the GUI on a selected point of the at least the portion of the image slice.

12. The method of claim 11, further comprising:
converting the one or more of the single mouse click and touch input into a 2D image by placing a 2D Gaussian point around the selected point.

13. The method of claim 12, wherein the 2D Gaussian point has a radius of approximately 10 pixels.

14. The method of claim 1, further comprising:
displaying the at least the portion of the image slice and the revised contour on the GUI; and
upon determining that the revised contour requires further revision, generating a second revised contour by:
receiving a second input from the user to the GUI to indicate a second point of revision,
inputting the one or more medical images, the first input, the second input, and the revised contour into the trained deep neural network that automatically extracts learned image characteristics,
processing the extracted learned image characteristics using the one or more deep-learning segmentation algorithms of the trained deep neural network, and
automatically generating the second revised contour using the processed extracted learned image characteristics.

15. The method of claim 14, wherein the second input comprises one or more of a single mouse click and a touch input to the GUI on a selected point of the at least the portion of the image slice.

16. The method of claim 15, further comprising:
converting the one or more of the single mouse click and the touch input into a 2D image by placing a 2D Gaussian point around the selected point.

17. The method of claim 14, further comprising:
displaying the at least the portion of the image slice and the second revised contour on the GUI.

18. The method of claim 17, further comprising:
receiving input from to the GUI the user accepting the second revised contour.

19. The method of claim 17, further comprising:
upon determining that the second revised contour requires further revision, repeating the generating and displaying steps.

20. The method of claim 1, further comprising:
displaying the at least the portion of the at least the portion of the image slice and the revised contour on the GUI; and
receiving input to the GUI accepting the revised contour.

21. The method of claim 1, further comprising:
propagating one or more additional initial contours in one or more additional image slices using the one or more deep-learning segmentation algorithms based on the revised contour.

22. The method of claim 1, further comprising:
updating the one or more deep-learning segmentation algorithms based the generating the revised contour.

23. The method of claim 22, wherein the updating is done at predetermined time intervals.

24. A system for computer-assisted contour revision in medical image segmentation, comprising:
a processor; and
a memory operatively coupled to the processor and configured to store computer-readable instructions that, when executed by the processor, cause the processor to:
select an image slice from one or more medical images of a patient, the image slice comprising an initial contour of a target anatomical structure in the one or more medical images;
display at least a portion of the image slice and the initial contour on a graphical user interface (GUI); and
upon a user determining that the initial contour requires revision, generate a revised contour by:
receiving a first input from a user to the GUI to indicate a first point of revision,
inputting the one or more medical images, the first input, and the initial contour into a trained deep neural network that automatically extracts learned image characteristics,
processing the extracted learned image characteristics using one or more deep-learning segmentation algorithms of the trained deep neural network, and
automatically generating the revised contour using the processed extracted learned image characteristics.

25. The system of claim 24, wherein the one or more medical images comprise three-dimensional (3D) images.

26. The system of claim 24, wherein the image slice comprises a two-dimensional (2D) image.

27. The system of claim 24, wherein the initial contour is generated by an external system.

28. The system of claim 24, wherein the computer-readable instructions, when executed by the processor, further cause the processor to:
input the one or more medical images into the trained deep neural network; and
automatically generate the initial contour using the one or more deep-learning segmentation algorithms to process the extracted learned image characteristics.

29. The system of claim 28, wherein the computer-readable instructions, when executed by the processor, further cause the processor to:
automatically generate one or more of an uncertainty value and a quality value for the initial contour.

30. The system of claim 29, wherein the selecting the image slice is done automatically based on, at least, one or more of the one or more of the uncertainty value and the quality value.

31. The system of claim 29, wherein the computer-readable instructions, when executed by the processor, further cause the processor to:
display the one or more of the uncertainty value and the quality value on the GUI.

32. The system of claim 24, wherein the computer-readable instructions, when executed by the processor, further cause the processor to:
display the one or more medical images on the GUI; and
receive user input to the GUI to generate the initial contour.

33. The system of claim 24, wherein the selecting the image slice is done manually via user input to the GUI.

34. The system of claim 24, wherein the first input comprises one or more of a single mouse click and a touch input to the GUI on a selected point of the at least the portion of the image slice.

35. The system of claim 34, wherein the computer-readable instructions, when executed by the processor, further cause the processor to:
convert the one or more of the single mouse click and touch input into a 2D image by placing a 2D Gaussian point around the selected point.

36. The system of claim 35, wherein the 2D Gaussian point has a radius of approximately 10 pixels.

37. The system of claim 24, wherein the computer-readable instructions, when executed by the processor, further cause the processor to:
display the at least the portion of the image slice and the revised contour on the graphical user interface (GUI); and
upon determining by the user that the revised contour requires further revision, generate a second revised contour by:
receiving a second input from the user to the GUI to indicate a second point of revision,
inputting the one or more medical images, the first input, the second input, and the revised contour into the trained deep neural network that automatically extracts learned image characteristics,
processing the extracted learned image characteristics using the one or more deep-learning segmentation algorithms of the trained deep neural network, and
automatically generating the second revised contour using the processed extracted learned image characteristics.

38. The system of claim 37, wherein the second input comprises one or more of a single mouse click and a touch input to the GUI on a selected point of the at least the portion of the image slice.

39. The system of claim 38, wherein the computer-readable instructions, when executed by the processor, further cause the processor to:
convert the one or more of the single mouse click and the touch input into a 2D image by placing a 2D Gaussian point around the selected point.

40. The system of claim 37, wherein the computer-readable instructions, when executed by the processor, further cause the processor to:
display the at least the portion of the image slice and the second revised contour on the GUI.

41. The system of claim 40, wherein the computer-readable instructions, when executed by the processor, further cause the processor to:
receive input from to the GUI the user accepting the second revised contour.

42. The system of claim 40, wherein the computer-readable instructions, when executed by the processor, further cause the processor to:
upon the user determining that the second revised contour requires further revision, repeat the generating and displaying steps.

43. The system of claim 24, wherein the computer-readable instructions, when executed by the processor, further cause the processor to:
display the at least the portion of the at least the portion of the image slice and the revised contour on the GUI; and
receive input from to the GUI accepting the revised contour.

44. The system of claim 24, wherein the computer-readable instructions, when executed by the processor, further cause the processor to:
revise one or more additional initial contours in one or more additional image slices using the one or more deep-learning segmentation algorithms based on the revised contour.

45. The system of claim 24, wherein the computer-readable instructions, when executed by the processor, further cause the processor to:
update the one or more deep-learning segmentation algorithms based on, at least, the generating the revised contour.

46. The system of claim 45, wherein the updating is done at predetermined time intervals.

47. A method for computer-assisted contour selection in medical image segmentation, comprising:
receiving one or more one image slices of a medical image of a patient, each of the one or more image slices comprising an initial contour of a target anatomical structure in the medical image;
inputting the one or more image slices into a trained deep neural network that automatically extracts learned image characteristics,
processing the extracted learned image characteristics using one or more deep-learning segmentation algorithms of the trained deep neural network,
automatically selecting at least a portion of the one or more image slices for review; and
displaying the at least the portion of the one or more image slices on a graphical user interface (GUI).

48. The method of claim 47, further comprising:
updating the one or more deep-learning segmentation algorithms based on, at least, one or more of the one or more images slices, the initial contours, and the at least a portion of the one or more image slices.

49. The method of claim 48, wherein the updating is done at predetermined time intervals.

50. A method for computer-assisted contour propagation in medical image segmentation, comprising:
receiving an image slice from one or more image slices of a medical image of a patient, the image slice comprising revisions to an initial contour of a target anatomical structure in the medical image;
inputting the image slice and the one or more image slices into a trained deep neural network that automatically extracts learned image characteristics,
processing the extracted learned image characteristics using one or more deep-learning segmentation algorithms of the trained deep neural network, and
automatically propagation one or more contours in the one or more image slices based on the revisions to the initial contour of the image slice.

51. The method of claim 50, further comprising:
updating the one or more deep-learning segmentation algorithms based on, at least, one or more of the one or more images slices, the initial contours, and the revisions to the one or more initial contours.

52. The method of claim 51, wherein the updating is done at predetermined time intervals.

53. The method of claim 50, wherein the automatically revising is based on one or more of a quality and uncertainty value of the initial contour of the image slice.
